(19) United States
(12) Patent Application Publication
(10) Pub. No.: US 2017/0007428 A1
(43) Pub. Date: Jan. 12, 2017

(54) ANTEGRADE FLOW MAINTENANCE SHEATH

(71) Applicant: Cook Medical Technologies LLC, Bloomington, IN (US)

(72) Inventor: Elizabeth A. Eaton, Bloomington, IN (US)

(21) Appl. No.: 14/795,511

(22) Filed: Jul. 9, 2015

(51) Int. Cl. A61F 2/856; A61F 2/95; A61M 25/01; A61M 39/06; A61M 27/00

(52) U.S. Cl. CPC A61F 2/856; A61M 39/06; A61M 27/002; A61M 25/0108; A61F 2/95; A61M 2025/0008

(57) ABSTRACT

The present embodiments relate to an endovascular treatment system comprising a sheath having a sheath lumen and a sheath aperture located on a circumferential surface of the sheath, and an internal liner disposed at least partially within the sheath lumen. The internal liner comprises a liner aperture, a protrusion, and a liner lumen, and has a delivery state and an expanded state. In the expanded state, the protrusion extends out of the sheath lumen through the sheath aperture.

### BACKGROUND

The embodiments relate to an endovascular treatment system and more particularly to a sheath and an internal liner used on such systems to maintain blood perfusion distal to an access site.

During endovascular procedures, it is often necessary to occlude a blood vessel for a period of time during which access is achieved, devices are positioned, and implants are delivered. During the time that the blood vessel is occluded, distal blood flow is minimal or non-existent, as much or all of the normal blood flow enters the endovascular treatment system and is directed out of the body or diverted into another system. This can lead to complications, particularly in cases that take a very long time (due to either expected procedural complexity or unexpected complications).

In extreme cases, the physician may have to balance the risk to the distal tissues with the more emergent need to keep the patient alive and complete the case. In order to combat this in cases where it may be expected that the distal tissue ischemia will be extensive, some physicians may rig up a sort of external bypass that may be cumbersome, inelegant, time consuming, and materials-dependant.

### BRIEF SUMMARY

The present embodiments relate to an endovascular treatment system comprising a sheath having a sheath lumen and a sheath aperture located on a circumferential surface of the sheath, and an internal liner disposed at least partially within the sheath lumen. The internal liner comprises a liner aperture, a protrusion, and a liner lumen, and has a delivery state and an expanded state. In the expanded state, the protrusion extends out of the sheath lumen through the sheath aperture.

The system may further comprise a hub at a distal end of the sheath. The sheath aperture may be located a set distance from the hub. The sheath may comprise a plurality of sheath markings. In some embodiments, the internal liner comprises a plurality of liner markings.

The protrusion may comprise an atraumatic characteristic, wherein the atraumatic characteristic is selected from the group consisting of a soft material, a flexible material, or a rounded edge. The sheath aperture may be radiopaque.

In one embodiment, the system further comprises a second sheath aperture located on the circumferential surface of the sheath. Further, in some embodiments, the protrusion may comprise a tip tapering in towards the internal liner.

### BRIEF DESCRIPTION OF THE DRAWINGS

The embodiments can be better understood with reference to the following drawings and description. The components in the figures are not necessarily to scale, emphasis instead being placed upon illustrating the principles of the embodiments. Moreover, in the figures, like referenced numerals designate corresponding parts throughout the different views.

FIG. 1 is an operational view of part of an endovascular treatment system, incorporating a sheath and an internal liner, in a blood vessel.

FIG. 2 is a perspective view of part of the endovascular treatment system of FIG. 1, with the sheath and the internal liner separated for illustrative purposes.

FIGS. 3A-3D are operational views of the endovascular treatment system of FIGS. 1-2, incorporating the sheath and the internal liner, opening and closing in a blood vessel.

FIGS. 4A-4B are operational views of an alternative embodiment of an endovascular treatment system, incorporating a sheath and an internal liner, exiting out of a hemostatic valve.

### DETAILED DESCRIPTION OF THE DRAWINGS AND THE PRESENTLY PREFERRED EMBODIMENTS

In the present application, the term “proximal” refers to a direction that is generally closest to the heart during a medical procedure, while the term “distal” refers to a direction that is furthest from the heart during a medical procedure.

Endovascular treatment systems are medical devices manipulated by a user and used to introduce instruments into blood vessels, such as prosthesis deployment systems that deploy prostheses within lumens in the human body.

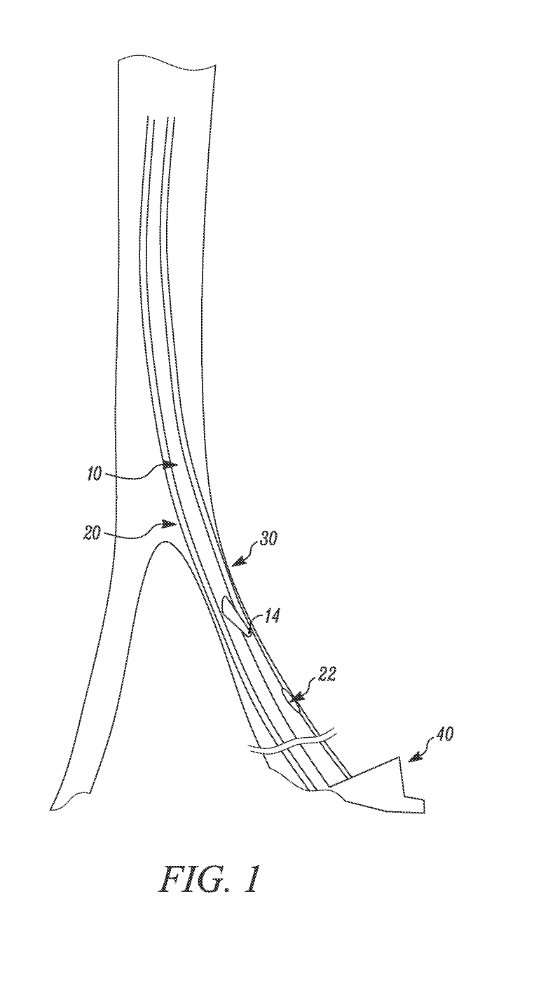

In FIGS. 1-4B, a part of the endovascular treatment system is shown and may include an internal liner 10 and a sheath 20. The sheath 20 may surround at least a portion of the internal liner 10. The sheath 20 may be coaxial with the internal liner 10.

Referring to FIG. 1, a part of an endovascular treatment system is shown in a blood vessel 30. The proximal end of the blood vessel 30 is located at the top of FIG. 1, and the distal end of the blood vessel 30 is located at the bottom of FIG. 1. The sheath 20 may serve as a stand-alone introducer or as part of a more complex delivery system. The sheath 20 may include a sheath aperture 22 at a set distance from a hub 40. The distance between the sheath aperture 22 and the hub 40 may vary. The internal liner 10 may be preloaded into the sheath 20 or loaded into the sheath 20 during a procedure.

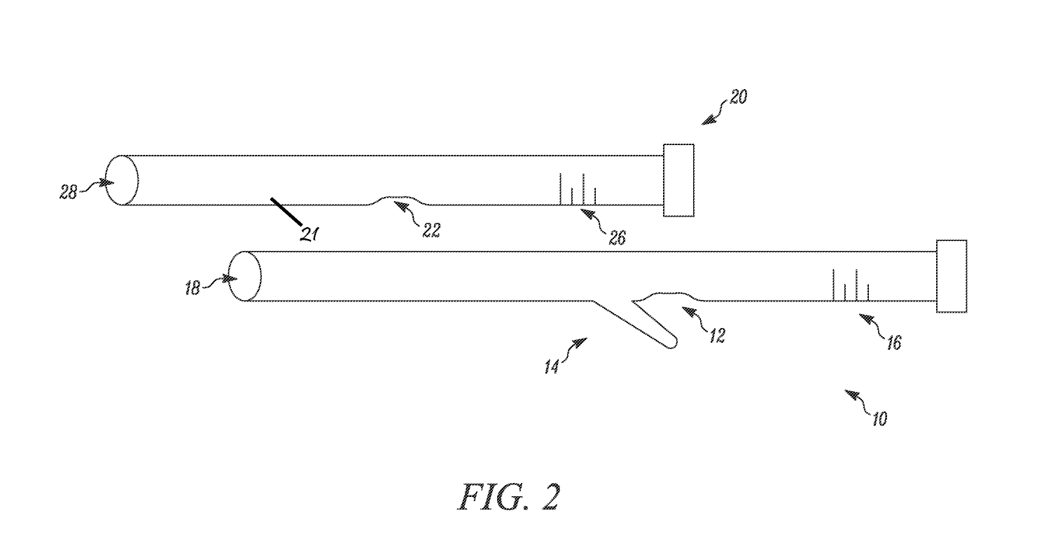

Referring to FIG. 2, the sheath 20 may include the sheath aperture 22 located on a circumferential surface 21 of the sheath 20, a plurality of sheath markings 26, and a sheath lumen 28. The plurality of sheath markings 26 may comprise printed lines, etchings, or other conventional indicators. The plurality of sheath markings 26 may be made radiopaque by any of the various means known in the art. The internal liner 10 may include a liner aperture 12, a protrusion 14, a plurality of liner markings 16, and a liner lumen 18. The protrusion 14 may be any shape; may be comprised of atraumatic characteristics, such as soft materials, flexible materials, and/or rounded edges; and may have a tendency to open outward when possible. The plurality of liner markings 16 may comprise printed lines, etchings, or other conventional indicators. The plurality of liner markings 16 may be made radiopaque by any of the various means known in the art.

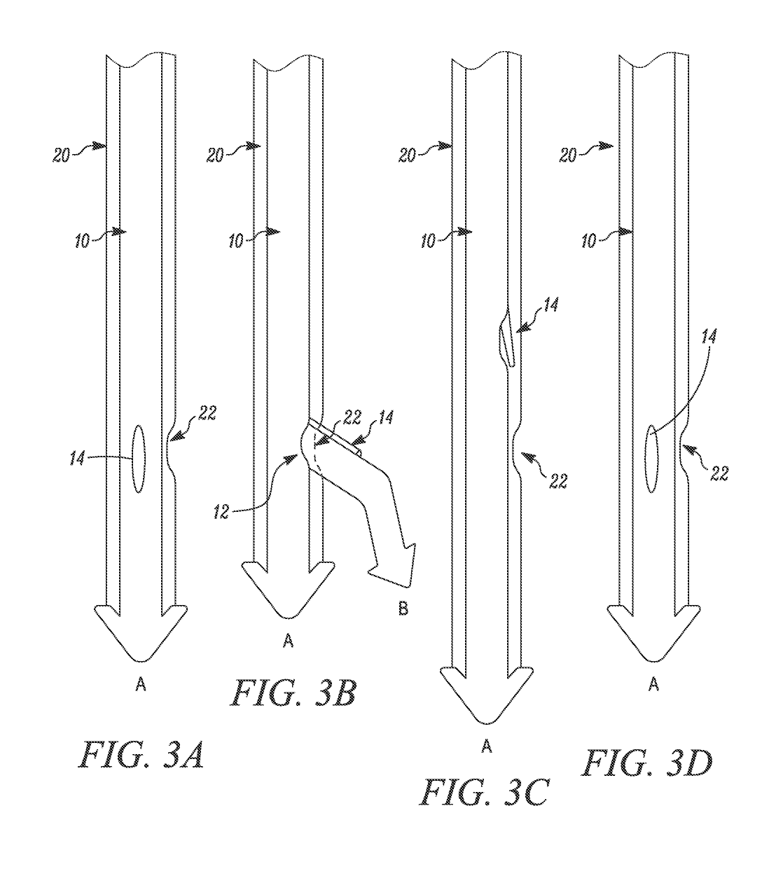

An exemplary endovascular treatment system, including the internal liner 10 and the sheath 20, is shown in FIGS. 3A-3D. Access to the blood vessel 30 may be obtained via the Seldinger technique or other conventional techniques in order to introduce a needle or a wire into the blood vessel 30. If the internal liner 10 is not preloaded into the sheath 20, it may be loaded into the sheath 20 to achieve the position shown in FIG. 3A. When the internal liner 10 is inserted into the sheath 20, the sheath lumen 28 may be coaxially in-between the internal liner 10 and a solid perimeter surface of the sheath 20. Also, when the internal liner 10 is inserted into the sheath 20, a dilator may be used inside the internal liner 10 in a conventional manner and then removed in a conventional manner before proceeding. The internal liner 10 may be locked with respect to the sheath 20 by conventional locking means, such as snap locks.

The internal liner 10, in a delivery state, and the sheath 20 may be positioned in the blood vessel 30. Once this position is achieved, the plurality of sheath markings 26 may be used to determine where the sheath aperture 22 is located within the blood vessel 30. Additionally, the sheath aperture 22 may be made radiopaque by any of the various means known in the art, to aid in lining up the sheath aperture 22 with necessary anatomical locations. At this point, the protrusion 14 may not be aligned with the sheath aperture 22, and the internal liner 10 may block the sheath aperture 22. This may cause blood to flow through the liner lumen 18 and out of the body or into another system. Blood flow from the proximal end of the endovascular treatment system to the distal end of the endovascular treatment system is shown by arrow A.

The internal liner 10 may then be unlocked and moved relative to the sheath 20 such that the protrusion 14 moves towards the sheath aperture 22. The plurality of liner markings 16 may be used to determine the relative position of the protrusion 14 and the sheath aperture 22. In an expanded state, the protrusion 14 of the internal liner 10 may then be aligned with the sheath aperture 22, and the protrusion 14 may pop out of the sheath 20 and point distally, as shown in FIG. 3B. A user may be able to feel that the protrusion 14 and the sheath aperture 22 are properly aligned instead of requiring additional contrast to confirm. This alignment may open a blood flow path, shown by arrow B, between the liner lumen 18 and the distal end of the blood vessel 30 through the liner aperture 12 and the sheath aperture 22. The protrusion 14 may direct blood flow to the distal end of the blood vessel 30 and may shield the wall of the blood vessel 30 from a potentially high pressure jet of blood coming out orthogonally to the wall of the blood vessel 30. The internal liner 10 may again be locked with respect to the sheath 20 by conventional locking means, such as snap locks. Other devices, such as stents, wires, or catheters, may be introduced into the blood vessel 30 as needed through the internal liner 10 and the sheath 20.

To close the blood flow path between the liner lumen 18 and the distal end of the blood vessel 30, the internal liner 10 may be advanced relative to the outer sheath 20 to retract the protrusion 14 back through the sheath aperture 22, as shown in FIG. 3C. Again, the plurality of liner markings 16 may be used to determine the relative position of the protrusion 14 and the sheath aperture 22. Both the internal liner 10 and the sheath 20 may then be retracted as a single unit out of the blood vessel 30 in a conventional manner. Alternatively, the internal liner 10 may be rotated relative to the sheath 20, as shown in FIG. 3D. The plurality of liner markings 16 and the plurality of sheath markings 26 may be used to confirm sufficient rotation between the internal liner 10 and the sheath 20. The internal liner 10 may then be retracted out of the blood vessel 30 relative to the sheath 20 in a conventional manner, or both the internal liner 10 and the sheath 20 may then be retracted as a single unit out of the blood vessel 30 in a conventional manner.

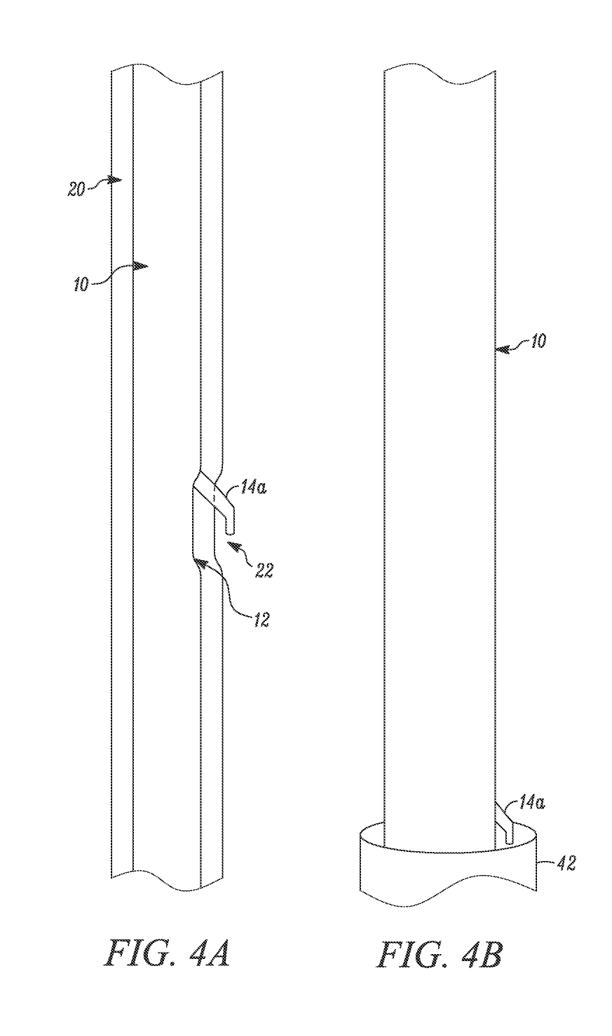

Another exemplary endovascular treatment system, including the internal liner 10 and the sheath 20, is shown in FIGS. 4A-4B. The same positioning of the internal liner 10 and the sheath 20 discussed above may apply, but an altered protrusion 14a may be added to facilitate withdrawal of the internal liner 10, in a retracted state, through a check flow hemostasis valve 42. The distal tip of the altered protrusion 14a may be formed at an angle to the proximal region of the protrusion, tapering in towards the body of the internal liner 10, as shown in FIG. 4A. Alternatively, the distal tip of the altered protrusion 14a may be curved in towards the body of the internal liner 10. When resistance (as from the check flow hemostasis valve 42) is encountered, the tip of the altered protrusion 14a may preferentially angle inward, thereby securing the altered protrusion 14a as it passes backwards through the check flow hemostasis valve 42, as shown in FIG. 4B.

Advantageously, the blood flow path between the liner lumen 18 and the distal end of the blood vessel 30 may prevent distal tissue ischemia, and the internal liner 10 and the sheath 20 may accomplish this without any cumbersome, inelegant, time consuming, or materials-dependant external bypass. Another advantage is that the internal liner 10 may be preloaded into the sheath 20 in order to reduce the complexity of a procedure.

An additional advantage is the selective imaging of various blood vessels. Currently, a user may place the sheath 20 just proximal to the target blood vessel 30 and may then shoot a contrast/saline mixture through the sheath 20 such that it empties out at the end of the sheath 20 and runs down into the target blood vessel 30, allowing for a clear image of the lumen of the blood vessel 30. To reduce the amount of contrast needed, the user may load a syringe with saline first, then contrast/saline, knowing that the saline portion at the back of the syringe will serve to push the contrast portion up the sheath 20. By allowing the sheath 20 to be positioned with sheath apertures near various target blood vessels, a user may push only as much volume as is needed to get to the sheath apertures. Backed by pure saline or not, this may be a smaller injection volume. The contrast may then run out of the sheath aperture 22 and demonstrate continued perfusion or show the lumen of the blood vessel 30. Lastly, withdrawal of the internal liner 10 through the check flow hemostasis valve 42 may allow for easy widening of the sheath lumen 28.

While only one sheath aperture 22 is depicted, multiple sheath apertures located on the circumferential surface 21 of the sheath 20 may be used if needed. For example, a user may selectively open and close distal blood flow in a variety of patient anatomies based on the circumstances of a procedure. In the end, the protrusion 14 may interact with each sheath aperture 22 in a similar fashion.

Additionally, the internal liner 10 need not be as long as the sheath 20. The internal liner 10 may be just long enough to extend from the hub 40 past the most distal sheath aperture 22 by one or two radii of sheath aperture 22. This may allow the internal liner 10 to be moved relative to the sheath 20 without causing any change in the length of the endovascular treatment system, which may be governed by the longer sheath 20.

While various embodiments have been described, the embodiments are not to be restricted except in light of the attached claims and their equivalents. Moreover, the advantages described herein are not necessarily the only advantages of the embodiments, and it is not necessarily expected that every embodiment will achieve all of the advantages described.

I claim:

1. An endovascular treatment system, comprising:
a sheath comprising a sheath lumen and a sheath aperture located on a circumferential surface of the sheath; and
an internal liner disposed at least partially within the sheath lumen, comprising a liner aperture, a protrusion selectively covering a portion of the liner aperture, and a liner lumen, and having a delivery state and an expanded state,
where, in the delivery state, the protrusion of the internal liner is disposed within the sheath lumen such that fluid flow through the sheath aperture is substantially inhibited, and
where, in the expanded state, at least a portion of the protrusion of the internal liner extends out of the sheath lumen through the sheath aperture such that fluid flow is permitted from the liner lumen through the liner aperture and the sheath aperture.

2. The endovascular treatment system of claim 1, further comprising a hub at a distal end of the sheath.

3. The endovascular treatment system of claim 2 wherein the sheath aperture is located a set distance from the hub.

4. The endovascular treatment system of claim 1 wherein the sheath comprises a plurality of sheath markings.

5. The endovascular treatment system of claim 1 wherein the internal liner comprises a plurality of liner markings.

6. The endovascular treatment system of claim 1 wherein the protrusion comprises an atraumatic characteristic, wherein the atraumatic characteristic is selected from the group consisting of a soft material, a flexible material, or a rounded edge.

7. The endovascular treatment system of claim 1 wherein the sheath aperture is radiopaque.

8. The endovascular treatment system of claim 1, further comprising a second sheath aperture located on the circumferential surface of the sheath.

9. The endovascular treatment system of claim 1 wherein the protrusion comprises a tip tapering in towards the internal liner.

10. An endovascular treatment system, comprising:
a sheath comprising a sheath lumen and a sheath aperture located on a circumferential surface of the sheath; and
an internal liner disposed at least partially within the sheath lumen, comprising a liner aperture, a protrusion selectively covering a portion of the liner aperture, and a liner lumen, and having a delivery state, an expanded state, and a retracted state, where, in the delivery state, the protrusion of the internal liner is disposed within the sheath lumen such that fluid flow through the sheath aperture is substantially inhibited, and
where, in the expanded state, at least a portion of the protrusion of the internal liner extends out of the sheath lumen through the sheath aperture such that fluid flow is permitted from the liner lumen through the liner aperture and the sheath aperture.
wherein the protrusion comprises a tip tapering in towards the internal liner, and
where, in the retracted state, the tip of the protrusion preferentially angles inward.

11. The endovascular treatment system of claim 10, further comprising a hub at a distal end of the sheath.

12. The endovascular treatment system of claim 11 wherein the sheath aperture is located a set distance from the hub.

13. The endovascular treatment system of claim 10 wherein the sheath comprises a plurality of sheath markings.

14. The endovascular treatment system of claim 10 wherein the internal liner comprises a plurality of liner markings.

15. The endovascular treatment system of claim 10 wherein the protrusion comprises an atraumatic characteristic, wherein the atraumatic characteristic is selected from the group consisting of a soft material, a flexible material, or a rounded edge.

16. The endovascular treatment system of claim 10 wherein the sheath aperture is radiopaque.

17. The endovascular treatment system of claim 1, further comprising a second sheath aperture located on the circumferential surface of the sheath.

18. A method for assisting endovascular treatment, comprising:
providing a sheath comprising a sheath lumen and a sheath aperture located on a circumferential surface of the sheath, and further providing an internal liner disposed at least partially within the sheath lumen and comprising a liner aperture, protrusion selectively covering a portion of the liner aperture, and a liner lumen;
positioning the sheath and the internal liner into a blood vessel;
moving the internal liner relative to the sheath such that the protrusion moves towards the sheath aperture; and
aligning the protrusion with the sheath aperture such that the protrusion extends out of the sheath lumen through the sheath aperture and points distally.

19. The method of claim 18, further comprising a step of moving the internal liner relative to the sheath such that the protrusion retracts back through the sheath aperture.

20. The method of claim 19, further comprising a step of withdrawing the internal liner through a check flow hemostasis valve.
